(19) United States
(12) Patent Application Publication
(10) Pub. No.: US 2022/0016285 A1
(43) Pub. Date: Jan. 20, 2022

(54) SYSTEMS AND APPARATUS FOR SANITIZING PERSONAL PROTECTIVE EQUIPMENT (PPE)

(71) Applicant: Clean Beam, LLC, Camp Douglas, WI (US)

(72) Inventors: Christopher Werfel, Streamwood, IL (US); Vadim Vain, Newmarket, Ontario (CA); Mark Cottone, St. Charles, IL (US)

(21) Appl. No.: 17/377,734

(22) Filed: Jul. 16, 2021

Related U.S. Application Data
(60) Provisional application No. 63/052,706, filed on Jul. 16, 2020.

(51) Int. Cl. A61L 2/10; A61L 2/24; A61L 2/26; B65G 47/248

(52) U.S. Cl. CPC A61L 2/10; A61L 2/24; A61L 2/26; B65G 47/248; B65G 2201/0229; A61L 2202/11; A61L 2202/122; A61L 2202/14; A61L 2202/26

(57) ABSTRACT

System and apparatus for sanitizing personal protective equipment (PPE) through the use of pulsed ultraviolet (PUV) light within a PUV light area configured to receive the PPE and having a light source to supply the PUV light. The system may further include a control mechanism in communication with the light source and operable to activate and deactivate the PUV light. A protective enclosure (for example, a housing and/or light curtains) houses the PPE and contains the PUV light within the PUV light area when the PUV light is activated. The PUV light has wavelengths in a range of 200 to 1100 nm.

### CROSS-REFERENCE TO RELATED APPLICATIONS

This application claims the benefit of U.S. Provisional Application No. 63/052,706 filed Jul. 16, 2020, the contents of which are incorporated herein by reference.

### BACKGROUND OF THE DISCLOSURE

The present disclosure generally relates to sanitizing personal protective equipment (PPE) including but not limited to respirators, masks, filters and face shields, and more particularly relates to a system incorporating a sanitization of PPE using pulsed ultraviolet (PUV) light.

Many facilities and field forces require individuals don various forms of personal protective equipment to reduce the potential spread of disease, virus, or infection within the facilities and to reduce the likelihood of person-to-person transmission. This may include, for example, hospitals, surgical rooms, laboratory clean rooms, food processing and production facilities, factories and other locations where it is of upmost importance to control pathogen transmission as well as field forces such as police, fire, paramedic, military and others who experience a high degree of person-to-person contact. Personal protective equipment (PPE), which is used herein to refer to, but is not limited to, respirators, masks, filters and face shields, are generally considered a single use item. In times of supply shortage and/or to reduce cost or waste, it may be desirable to sanitize and re-use certain PPE items multiple times subject to other disposal considerations such as soiling or excessive wear.

Some current methods for sanitizing PPE include fixed ultraviolet light (UV-C) and vaporized hydrogen peroxide, and various sterilizing liquids applied directly to the PPE, as well as heat treating. The UV-C light, when applied properly, is capable of reducing, if not substantially neutralizing, certain pathogens and other deleterious materials and substances that may be on surfaces of the PPE. The level of intensity of the UV-C light and the manner in which it is applied may make the sanitization process time consuming and inefficient for eliminating pathogens on PPE. Pulsed ultraviolet (PUV) light has the ability to inactivate pathogens at a DNA and RNA level. Similarly, vaporized hydrogen peroxide sanitization methods are capable of reducing, if not substantially neutralizing, certain pathogens and other deleterious materials and substances that may be on surfaces of the PPE. The amount of time required to effectively sanitize PPE using vaporized hydrogen peroxide is significantly longer and less efficient than the application of PUV.

In view of the above, it can be appreciated that there are certain problems, shortcomings or disadvantages associated with existing methods for sanitizing PPE.

### SUMMARY OF THE DISCLOSURE

Disclosed is a system for sanitizing PPE using PUV light. PUV light is UV light that is pulsed in part to increase the effective amount of energy applied at one time, yet reducing the dwell time of the UV light. PUV light is generally applied from a high intensity light source. The invention disclosed herein may use PUV as a sanitizing agent for the application of a broad band wave light having wavelengths in a range of about 200 to about 1100 nm. This light may be capable of a reduction in pathogens including bacteria and viruses.

According to one aspect, a system for sanitizing personal protective equipment (PPE) includes a pulsed ultraviolet (UV) light area having a light source to supply pulsed UV (PUV) light and configured to receive the PPE. The system further includes a control mechanism in communication with the light source. The control mechanism is operable to activate and deactivate the PUV light. A protective enclosure (for example, a housing and/or light curtains) houses the PPE and contains the PUV light within the PUV light area when the PUV light is activated. The PUV light has wavelengths in a range of 200 to 1100 nm and is pulsed at a constant energy per pulse.

In an exemplary but nonlimiting embodiment of the system, an operator loads PPE onto an area that uses PUV light to eliminate pathogens from the PPE. For example, the operator may load PPE onto a loading area that moves the PPE via a conveyor system into a PUV light area comprising a light shielded area preventing the PUV light from escaping once it is activated. Preventing the PUV light from escaping is desirable as the light may have harmful effects to the eyes and/or skin of the operator or bystanders. One manner in which the PUV light may be applied is a button that when pushed may commence an automated timed cycle that may be predetermined based on the pathogen reduction desired. The pathogen reduction may be determined based on the requirements of the particular facility or operator using the system. The automated timed cycle may be electronically programmable, and may be remotely programmable through any wired or wireless connection. In an exemplary but nonlimiting embodiment, the PUV light may run and may comprise multiple PUV lights that remain stationary or one PUV light that may move on a tracking system to cover the entirety of the PPE.

In an exemplary but nonlimiting embodiment, the system may comprise a loading area, one or multiple lanes, a conveyor system, a mechanism to rotate the PPE, a PUV light area and an offloading area. As an example, a conveyor system may comprise a series of moving rods which advance the PPE items through the various areas. As an example, the PUV light area may be defined or contained within a protective enclosure that the PPE is conveyed into which houses the PUV light source, which is either over or under the conveyor (if the conveyor is transparent), or it could be located on one side. A “flipping” mechanism is therefore employed, so as to rotate (e.g., flip) the PPE article, such as a mask as it passes the PUV light source. All sides of the article are preferably capable of being exposed using a single PUV source located relative to the conveyor (for example, above the conveyor). As another example, the protective enclosure may also contain reflective panels (for example, stainless steel or mirrored side panels) that reflect the PUV light within the protective enclosure for more efficient exposure. In an exemplary but nonlimiting embodiment, the system may automatically shut off exposing a load of PPE after a predetermined amount of time passes.

In one form of the invention, an apparatus for sanitizing personal protective equipment (PPE) has a conveyor upon which PPE is received for movement through the apparatus, the PPE presenting a first surface when emplaced on the conveyor. A source of sanitizing electromagnetic energy is located so as to irradiate a sanitizing area of the conveyor upon which PPE is received. A PUV light or lights is on such source. A PPE manipulation mechanism is associated with the conveyor in the vicinity of the sanitizing area, the manipulation mechanism engaging with the PPE after the first surface of the PPE has been subjected to the sanitizing energy and reorienting the PPE to thereby expose a second (e.g., lower) surface of the PPE to the sanitizing energy. A control mechanism, in communication with the conveyor and sanitizing energy, is provided, that activates and deactivates the PUV light and operates the conveyor.

In a preferred aspect, the manipulation mechanism comprises a flipper device having at least a pair of panels hinged together along a pivot, and a rotary device engaging the flipper device to pivot the panels, PPE being emplaced upon one of the panels to expose the first surface and then being reoriented by the flipper device onto a second panel to expose a different (second) surface of the PPE. The rotary device may be a stepper motor, the pivot having an element engaged by the stepper motor to rotate the pivot about a pivot axis and move the one panel through an arc and move the PPE to the second panel.

These and other aspects, advantages, applications and features of the disclosure will be further understood upon consideration of the following detailed description, taken in conjunction with the drawings.

### BRIEF DESCRIPTION OF THE DRAWINGS

For the purpose of facilitating an understanding of the subject matter sought to be protected, there are illustrated in the accompanying drawings embodiments thereof, from an inspection of which, when considered in connection with the following description, the subject matter sought to be protected, its construction and operation, and many of its advantages should be readily understood and appreciated.

FIG. 1 is a perspective top view schematically representing a nonlimiting embodiment of a system for sanitizing PPE.

FIG. 2 is a schematic detail view schematically representing a nonlimiting embodiment of a manipulation mechanism capable of use in the system of FIG. 1.

FIG. 3 is a side view schematically representing the embodiment of FIG. 1 including detail of a nonlimiting embodiment of a conveyor mechanism capable of use in the system of FIG. 1.

FIG. 4 is another schematic representation of the manipulation mechanism of FIG. 2.

FIG. 5 is a perspective view schematically representing a nonlimiting embodiment of a conveyor mechanism chassis capable of use in the system of FIG. 1.

FIG. 6 schematically represents a hinge that may be used with a flipper mechanism on a conveyor of an embodiment.

FIG. 7 is a top plan view of the hinge of FIG. 6.

FIG. 8 is a perspective view schematically representing some internal detail of a conveyor mechanism of the kind shown in FIG. 5.

### DETAILED DESCRIPTION

The intended purpose of the following detailed description of the invention and the phraseology and terminology employed therein is to describe what is shown in the drawings, which include the depiction of one or more nonlimiting embodiments of the invention, and to describe certain but not all aspects of what is depicted in the drawings, including the embodiment(s) depicted in the drawings. The following detailed description also identifies certain but not all alternatives of the embodiment(s) depicted in the drawings. Therefore, the appended claims, and not the detailed description, are intended to particularly point out subject matter regarded as the invention, including certain but not necessarily all of the aspects and alternatives described in the detailed description.

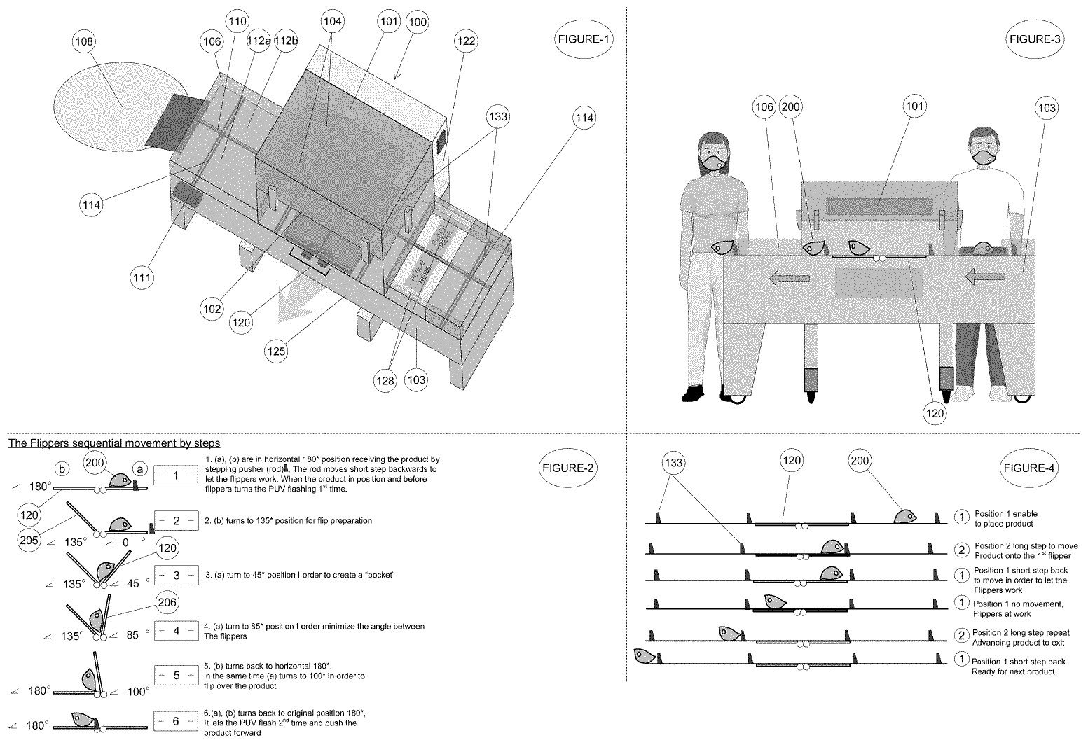

Turning now to the drawings, FIG. 1 depicts a perspective view of an embodiment of a PUV light system 100 for sanitizing PPE 200 (FIGS. 2 through 4). For purposes of illustration, and not by limitation, a mask type of PPE 200 is shown as the item(s) being sanitized. The system 100 may include a loading area 103, a PUV light area 101 with PUV light sources 104, an outfeed area 106, optional rotary collection table 108, separation bar 110 that serves to divide a conveyor of the conveyor mechanism to define two conveyor lanes 112a and 112b, a protective enclosure 102 (schematically represented as a box-like structure or housing, though could additionally or alternatively comprise light curtains), a conveyor mechanism 114, which will be described in more detail shortly which includes a manipulation or “flipping” mechanism 120, a control system and electrical cabinet 122; and a support structure or platform 125.

The manipulation mechanism 120 may be used on either or both conveyor lanes 112a and 112b to rotate, or flip, the conveyed PPE, such as a mask, which is traversing through the light area 101. FIG. 2 depicts a schematic view of one embodiment of the manipulation mechanism 120, which is also shown in somewhat similar fashion in FIG. 4. As evident from FIGS. 2 and 4, the manipulation mechanism 120 may be adapted to rotate the PPE 200 fully 180 degrees, though it is also within the scope of the invention that the PPE 200 could be rotated more or less than 180 degrees as the result of a single rotation or flip, to expose two or more different sides of the PPE 200 to PUV light emitted by the PUV light sources 104. The manipulation mechanism 120 will be described in more detail below.

In the system 100, an operator may first load PPE 200 into a loading area 103. As previously noted, the conveyor represented in the drawings has two lanes 112a and 112b defined by the separation bar 110 therebetween. The loading area may contain light curtains 128 to limit PUV light exposure from the PUV light area 101 to the operator(s). The PPE 200 may then be conveyed by a series of push rods 133 using a drive motor 111, for example, a stepping motor or the like, to drive the loop conveyor 114. From the loading area 103, the conveyor 114 moves the PPE 200 along and through the PUV light area 101 where the PUV light source 104 is activated. As will be described, the PPE 200 are flipped via the manipulation mechanism 120, whereupon the PUV light source 104 is activated a second time thereby sanitizing different sides (e.g., the top and bottom sides) of the PPE 200. When that is complete, the PPE 200 are conveyed into the outfeed area 106 and optional rotary collection table 108.

In the system 100, PPE 200 may first be loaded in the loading area dual lanes 112a and 112b and then conveyed to the PUV light area 101. The PUV light area 101 may contain a light source or element that supplies PUV light when activated by the system 100. Such a PUV light array is available from XENON Corporation and utilizes bulbs that are reported to emit UV light at wavelengths of about 200 to about 1100 nm, as well as optics and a mounting system. Other light sources may be substituted, of course, as those of skill will recognize.

In some embodiments, the system 100 may exclude the loading area 103, offload area 106, conveyor mechanism 114, and manipulation mechanism 120 and yet utilize a PUV light area 101 with the other elements. In this variant embodiment, the operator loads the PPE 200 directly into the PUV light area 101, closes the protective enclosure 102, activates the PUV light source 104, opens the protective enclosure 102, manually flips the PPE 200, closes the protective enclosure 102, activates the PUV light source 104, and then offloads the PPE 200.

Once the PPE 200 is loaded or conveyed into the PUV light area 101, the system 100 may be activated and the light source 104 in PUV light area 101 may be turned on to supply PUV light to the PPE 200 residing in the PUV light area 101. This may be achieved by the user pressing a button causing the activation or system 100 to automatically activate, for example, in response to one or more sensors (not shown) that generate a signal indicating that the PPE 200 is positioned in the PUV light area 101. For an amount of time, PUV light is emitted and projected onto the PPE 200 in the PUV light area 101. The amount of time may be predetermined depending on a desired pathogen reduction level or requirements of the facility and/or operator using the system 100. Further, the manipulation mechanism 120 represented in FIG. 2 may be configurable to be enabled, disabled or adjusted to the requirements of the facility and/or operator using the system 100.

The PUV light may be a broad band wave light having wavelengths in a range of about 200 to about 1100 nm. The protective enclosure 102 can prevent the PUV light from escaping and limit the light exposure to the operator that would cause damage, such as the operator's eyes or skin.

In an exemplary but nonlimiting embodiment, there may be reflective panels (for example, stainless steel or mirror panels) along the sides of the protective enclosure 102 to reflect the PUV light for a more effective exposure of the entirety of the PPE 200.

The electrical circuitry or control mechanism that controls the operation of system 100 may reside in a support structure and/or an electrical cabinet 122 represented in FIG. 1.

Turning now to the manipulation mechanism 120, FIGS. 6 through 8, reveal that the mechanism 120 may include two panels (for example, levers, flaps, or flippers) 205 and 206, which are hinged together in a common plane, as by one or more hinges 208. The panels 205 and 206 rest atop the conveyor surface 116, which may be constructed of a stainless steel, but again, substitutions are well known. At end edges of the panels 205 and 206 are the push rods 133.

Stepper motors 201 and 202 are located adjacent the conveyor surface 116, and within the light source area 101. The stepper motors 201, 202 are positioned so as to engage with wheels or disks 204a, 204b which are attached to the ends of the rod or spindle 210 of the hinge 208. The engagement and operation of the stepper motors with the flipper mechanism are illustrated in FIGS. 2 and 4. As shown therein, the respective panels 205 and 206 are caused to pivot, or raise up and down, in a sequence, represented in FIG. 2 as steps 1 through 6. FIG. 4 illustrates in a more elevational view without the pivoting action being shown (which is in FIG. 2), with the PPE 200 approached the area of the stepper motors 201 and 202, thence being rotated or flipped, and progress out. The push rods 133 serve to keep the PPE 200 relatively contained within the flipper mechanism 120.

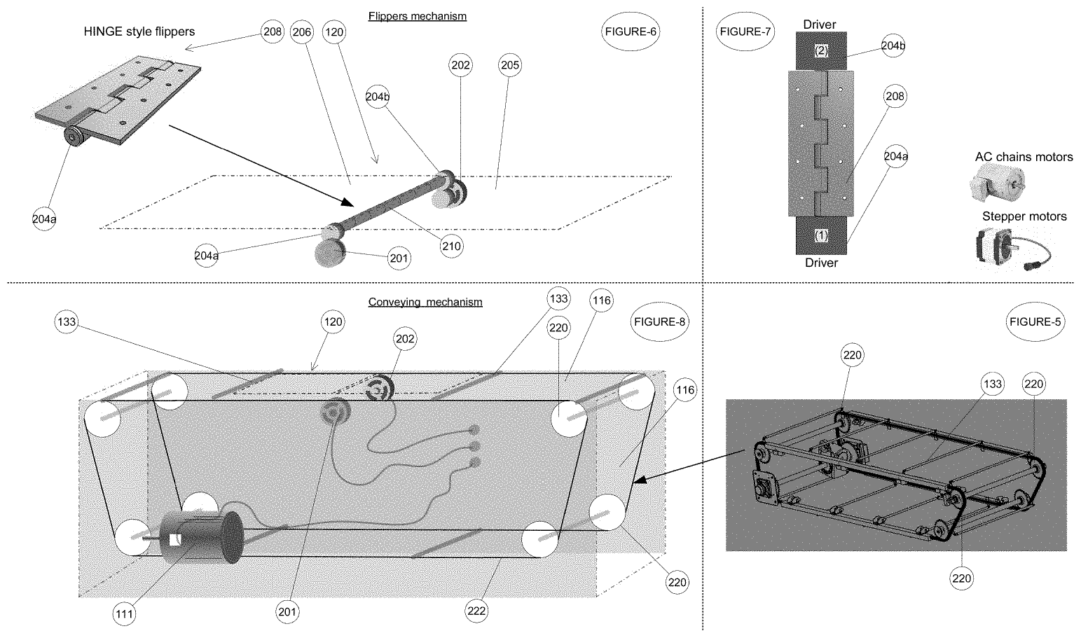

What is shown in FIG. 5 is a somewhat standard-type chassis for a conveyor capable of use with the system 100. The chassis has sprockets or wheels 220 at either end, with a chain 222 or other type loop drive that turns on drive sprockets associated with the drive motor 111. The conveyor can be conventional, and further details well known.

The present disclosure is thus considered to yield a PUV light sanitization system for sanitizing PPE by exposing the PPE to PUV light, in one exemplary but nonlimiting application of the disclosure. While this system has been described with respect to a particular embodiment(s), and application in a specific environment, those of skill will recognize modifications of components, elements, materials, arrangements, and the like which will still fall within the spirit and scope of the invention, and the invention is not to be limited to such embodiment(s) or specific details.

What is claimed is:

1. A system for sanitizing personal protective equipment (PPE), the system comprising:
a pulsed ultraviolet (UV) light area configured to receive the PPE and having a light source that emits pulsed UV (PUV) light;
a control mechanism in communication with the light source and operable to activate and deactivate the PUV light; and
a protective enclosure that houses the PPE and contains the PUV light within the PUV light area when the PUV light is activated;
wherein the PUV light is emitted at wavelengths in a range of 200 to 1100 nm and pulsed at a constant energy per pulse.

2. The system of claim 1, wherein the control mechanism deactivates the light source after the light source has been activated for a predetermined period of time and/or number of pulses.

3. The system of claim 1, further comprising a loading area for preparing the items of PPE to enter the PUV light area.

4. The system of claim 1, further comprising an outfeed area and rotary collection table to collect the sanitized PPE.

5. The system of claim 1, wherein the PUV light area contains the light source which is positioned in a manner to emit and project the PUV light onto the PPE.

6. The system of claim 5, wherein the protective enclosure comprises a housing that contains the light source, the PUV light area is within the housing, and the housing comprises reflective panels that reflect the PUV light within the housing.

7. The system of claim 1, wherein the control mechanism moves the PPE on a conveyor system.

8. The system of claim 1, wherein the light source includes two PUV light sources that direct PUV light towards the PPE.

9. The system of claim 1, wherein the protective enclosure, loading area, PUV light area and outfeed area include a separator bar to establish two lanes for processing the PPE.

10. The system of claim 1, wherein control mechanism includes a control adapted to modify an intensity and/or duration of application of the PUV light.

11. The system of claim 1, further comprising a manipulation mechanism that rotates the PPE thereby causing the PUV light to be applied to different sides of the PPE on a single sanitization pass.

12. A system for sanitizing personal protective equipment (PPE), the system comprising:
a pulsed ultraviolet (PUV) light area configured to receive the PPE and having a light source to supply PUV light;
a control mechanism in communication with the light source and operable to activate and deactivate the light source;
a conveyor system that moves the PPE from a loading area, through the PUV light area, and onto an outfeed area; and
a protective enclosure that houses the PPE and contains the PUV light within the PUV light area when the PUV light is activated, the enclosure containing the light source, being divided into two or more lanes, and containing a manipulation mechanism which rotates the PPE thereby allowing the PUV light to be applied to different sides of the PPE on a single sanitization pass, the light source being positioned in a manner to emit and project the PUV light onto the PPE;
wherein the PUV light is emitted at wavelengths in a range of 200 to 1100 nm.

13. An apparatus for sanitizing personal protective equipment (PPE), the apparatus comprising:
a conveyor upon which the PPE is received for movement through the apparatus, the PPE presenting a first surface thereof when emplaced on the conveyor;
a source of sanitizing electromagnetic energy which is located so as to irradiate a sanitizing area of the conveyor upon which PPE is received;
a manipulation mechanism associated with the conveyor in the vicinity of the sanitizing area, the manipulation mechanism engaging with the PPE after the first surface of the PPE has been subjected to the sanitizing energy and reorienting the PPE to thereby expose a second surface of the PPE to the sanitizing energy; and
a control mechanism, in communication with the conveyor and sanitizing energy, that activates and deactivates the PUV light and operates the conveyor.

14. The apparatus of claim 13, wherein the manipulation mechanism comprises a flipper device having panels hinged together along a pivot, and a rotary device engaging the flipper device to pivot the panels, PPE being emplaced upon one of the panels to expose the first surface and then being reoriented by the flipper device onto a second of the panels to expose the second surface.

15. The apparatus of claim 14, wherein the rotary device is a stepper motor, the pivot having an element engaged by the stepper motor to rotate the pivot about a pivot axis and move the one panel through an arc and move the PPE to the second panel.

16. The apparatus of claim 13, wherein the sanitizing energy is a light source of PUV light.

17. The apparatus of claim 15, wherein the sanitizing energy is a light source of PUV light.
